(19) United States
(12) Patent Application Publication
(10) Pub. No.: US 2009/0176875 A1
(43) Pub. Date: Jul. 9, 2009

(54) METHOD FOR MANUFACTURING METAL NANO PARTICLE SOLUTION

(76) Inventors: Dong-Pyo Kim, Ansan-si (KR); Yoon-Hyuck Choi, Yongin-si (KR); Choong G. Row, Moreland Hills, OH (US)

Correspondence Address: PARK LAW FIRM, 3255 WILSHIRE BLVD, SUITE 1110, LOS ANGELES, CA, 90010, US

(21) Appl. No.: 11/971,614

(22) Filed: Jan. 9, 2008

(51) Int. Cl. A61K 31/28; A61K 31/315; A61K 31/295; A61K 31/30

(52) U.S. Cl. 514/494; 514/499; 514/495; 514/501; 977/915; 977/904; 977/840

(57) ABSTRACT

A method for manufacturing a stabilized metal nano particle solution is disclosed. This method manufactures a metal nano particle solution so as to make a metal substance such as silver, gold, copper, zinc or cobalt into ultra-capsular nano particles. That is to say, this new method is simple and suitable for mass production without requiring any separate reducer putting process at a room temperature while a transition metal nano particle solution is produced. In this method, an alcohol solution including a metal salt solution and a soluble polymer is mixed at a room temperature to make a nano metal particle solution with a particle size of 100 nm or less.

### BACKGROUND OF THE INVENTION

1. Field of the Invention

The present invention relates to a method for manufacturing a stabilized metal nano particle solution, and more particularly to a method for manufacturing a metal nano particle solution, which allows to make metal substances such as silver, gold, copper, zinc and cobalt into ultra-capsular nano particles.

2. Description of the Related Art

Generally, nano particles with a size of 100 nm or less have significantly improved electrical and thermal properties in comparison to greater particles and also have antifungal ability due to improved reactivity, as well known in the art. Thus, various conductive inks, electromagnetic wave shields and antifungal agents have been made using the nano particles.

Meanwhile, for making a nano particle solution in the prior art, three methods have been generally applied to the present. First, various kinds of mill equipment are used to make particles of several millimeters or micrometers into a size of 100 nm or less in a mechanical manner. Second, metal is heated and evaporated into a gas, and then the gaseous metal is cooled to generate particulate powder of several nanometers. Third, a chemical manner is used to compose a metal nano particle solution in a solution state.

However, the conventional methods for manufacturing a metal nano particle solution require too much time and have complexity and unstable elements, which should be solved.

In particular, among the above three manners, the chemical manner mixes polyvinyl alcohol, polyvinyl pyrrolidone, or sodium dodecyl sulfate with a metal salt solution, and adds NaBH₄ or ascorbic acid thereto at a room temperature or an increased temperature as a reducer or irradiating radiant rays such as gamma rays to a stabilizer or a metal salt mixture solution in order to stabilize the nano particles. However, it has complicated reaction conditions or requires much cost for dealing dangerous radiant rays, and also it is lack of safety and substantially not allowing mass production, so measures for such problems are urgently needed.

### SUMMARY OF THE INVENTION

Therefore, the present invention has an essential point in providing a new method for simply manufacturing a metal nano particle solution suitable for mass production without needing any addition process of a separate reducer at a room temperature during the transition metal nano particle solution manufacturing procedure.

### BRIEF DESCRIPTION OF THE DRAWINGS

FIG. 1 is a TEM (Transmission Electron Microscope) photograph showing a reaction solution according to a first embodiment of the present invention;

FIG. 2 is a TEM photograph showing a reaction solution according to a second embodiment of the present invention;

FIG. 3 is a TEM photograph showing a reaction solution according to a third embodiment of the present invention;

FIG. 4 is an antifungal test report of the present invention; and

FIG. 5 is a graph showing the change of thermal conductivity according to the content of nano copper in the present invention.

### DESCRIPTION OF THE PREFERRED EMBODIMENT

The present invention will be described in more detail with reference to the accompanying drawings and following embodiments in order to accomplish the above object.

That is to say, the present invention is characterized in a method for manufacturing a nano particle solution in which a solution of a transition metal salt such as silver, gold, copper, zinc and cobalt is mixed or blended with alcohol, glycol or alcohol-glycol mixture including a stabilizer such as polyvinyl pyrrolidone.

If the above metal salt is mixed with soluble polymer and alcohol or glycol as mentioned above, metal ions of the metal salt are reacted with the alcohol or glycol, thereby reducing the metal ions. Then, metal particles reduced and lumped in a nano size are coupled with the soluble polymer to form a stabilized nano particle solution.

The metal salt includes a soluble metal salt generated by reacting a transition metal such as solver, copper, zinc and cobalt with acid such as nitric acid, sulphuric acid, hydrochloric acid and acetic acid. The stabilizer includes a soluble polymer such as polyvinyl pyrrolidone (PVP), polyvinyl alcohol and polyvinyl acetate. In addition, the alcohol includes methanol, ethanol, propyl alcohol and butyl alcohol.

### Embodiment 1

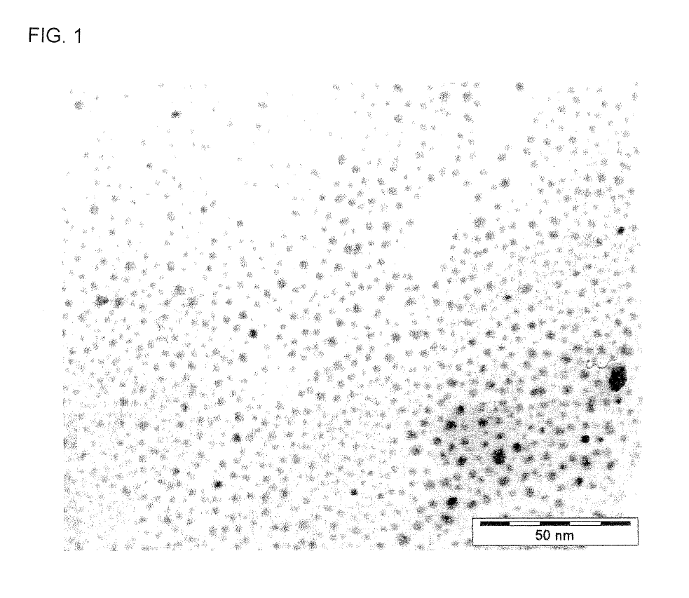

Two kinds of solutions were prepared: a solution in which 7.87 g of silver nitrate is melted in 20 g of water and a solution in which 5 g of PVP is melted in a mixture solution containing 10 g of ethanol and 50 g of poly ethyl glycol, and then the prepared two solutions were mixed and stirred at a room temperature. The reacted solution was photographed using TEM (Transmission Electron Microscope), and a nano silver solution having regular particle distribution with a particle size of 10 nm or less may be manufactured as shown in the photograph of FIG. 1.

### Embodiment 2

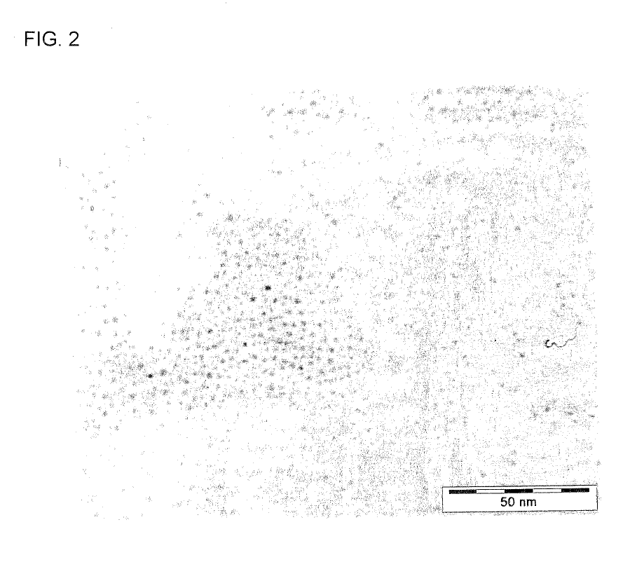

Two kinds of solutions were prepared: a solution in which 7.87 g of silver nitrate is melted in 20 g of water and a solution in which 6 g of PVP is melted in a mixture solution containing 100 g of isopropyl alcohol, and then the prepared two solutions were mixed and stirred at a room temperature. The reacted solution was photographed using TEM, and a nano silver solution having regular particle distribution with a particle size of 10 nm or less may be manufactured as shown in the photograph of FIG. 2.

### Embodiment 3

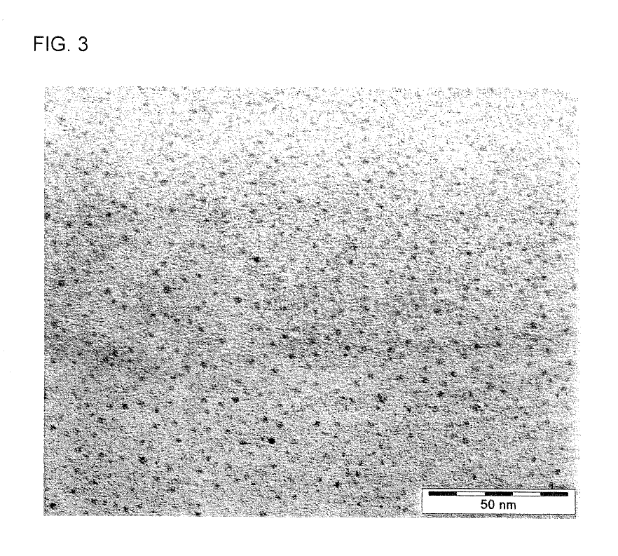

Two kinds of solutions were prepared: a solution in which 19.51 g of copper nitrate is melted in 20 g of water and a solution in which 9 g of PVP is melted in a mixture solution containing 10 g of ethyl alcohol, and then the prepared two solutions were mixed and stirred at a room temperature. The reacted solution was photographed using TEM, and a nano silver solution having regular particle distribution with a particle size of 10 nm or less may be manufactured as shown in the photograph of FIG. 3.

In the embodiments 4 and 5, zinc and cobalt is used instead of silver and copper of the embodiments 1 to 3, and their reaction states were experimented in the same way.

Antifungal property and heat transfer property of the nano solution particles manufactured according to the above method were evaluated as follows.

### Evaluation 1

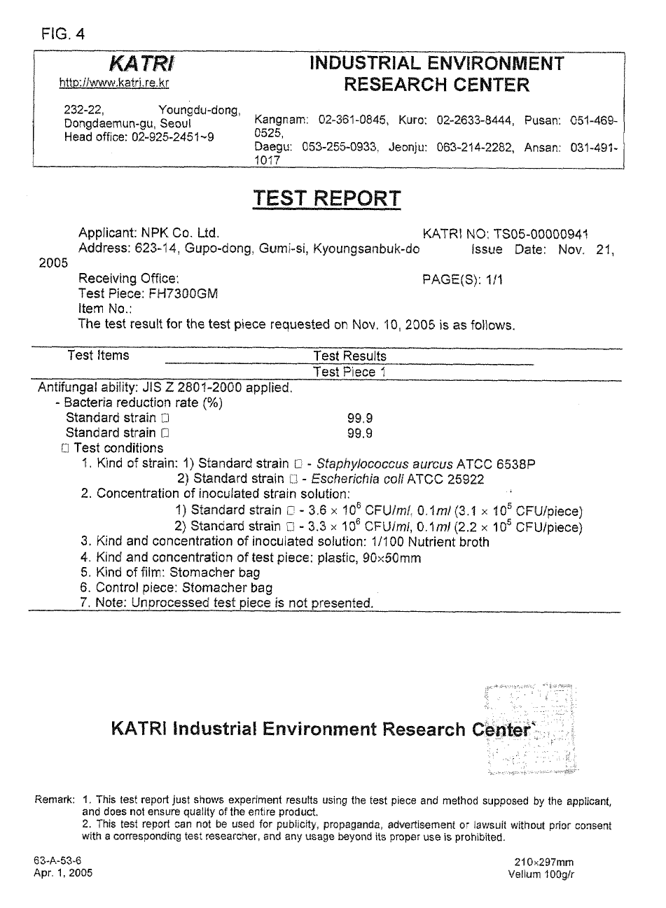

The solution manufactured in the embodiment 1 was diluted in water to make the nano silver particle content into 20 ppm, and then mixed with a complex polypropylene (PP) resin. Then, an antifungal ability was evaluated, and it showed 99.9% antifungal ability against staphylococcus and colon bacillus, respectively, as shown in FIG. 4.

### Evaluation 2

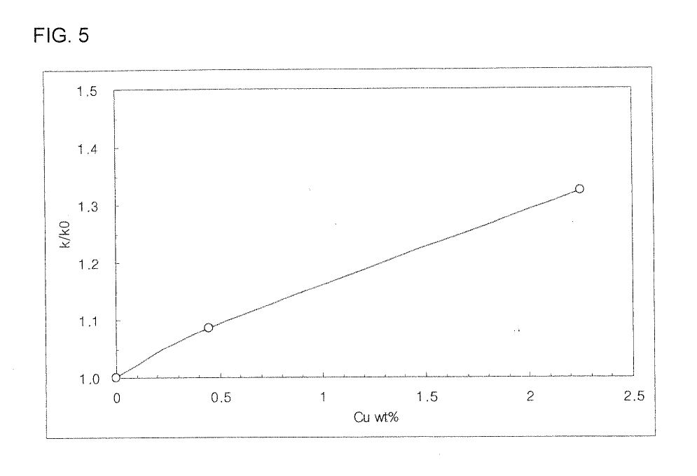

The solution manufactured in the embodiment 3 was diluted in ethyl glycol, and then a thermal conductivity was measured with changing the content of nano copper particles. As a result, the thermal conductivity was increased by 32% at the nano copper particle content of 2.3 wt % as shown in FIG. 5.

### APPLICABILITY TO THE INDUSTRY

As described above, the method for manufacturing a metal nano particle solution according to the present invention is capable of giving a metal nano particle solution with improved antifungal ability and thermal conductivity using a simple process of mixing a metal salt solution with an alcoholic solution including a stability, so it has many expectations.

What is claimed is:

1. A method for manufacturing a metal nano particle solution,
wherein a metal salt solution is mixed with an alcoholic solution including a soluble polymer to make a metal nano particle solution having a particle size of 100 nm or less.

2. The method for manufacturing a metal nano particle solution according to claim 1,
wherein the metal salt employs a metal selected from the group consisting of silver, copper, zinc and cobalt.

3. The method for manufacturing a metal nano particle solution according to claim 2,
wherein the metal salt is generated by reacting a metal selected from silver, copper, zinc and cobalt with an acid selected from nitric acid, sulphuric acid and hydrochloric acid.

4. The method for manufacturing a metal nano particle solution according to claim 1,
wherein the metal salt is generated by reacting a metal selected from silver, copper, zinc and cobalt with an acid selected from nitric acid, sulphuric acid and hydrochloric acid.

5. The method for manufacturing a metal nano particle solution according to claim 1,
wherein the alcoholic solution includes an alcohol selected from the group consisting of methyl alcohol, ethyl alcohol, propyl alcohol and butyl alcohol.

6. The method for manufacturing a metal nano particle solution according to claim 1,
wherein the alcohol solution employs glycol, poly glycol, or a mixture solution in which glycol or poly glycol is mixed with methyl alcohol, ethyl alcohol, propyl alcohol or butyl alcohol.

7. The method for manufacturing a metal nano particle solution according to claim 6,
wherein the poly glycol employs poly ethyl glycol.

8. The method for manufacturing a metal nano particle solution according to claim 1,
wherein the soluble polymer is selected from the group consisting of polyvinyl pyrrolidone, polyvinyl alcohol and polyvinyl acetate.
